(19) United States
(12) Patent Application Publication
(10) Pub. No.: US 2011/0311399 A1
(43) Pub. Date: Dec. 22, 2011

(54) APPARATUS AND METHOD FOR STERILIZING OBJECTS

(75) Inventors: Angelo Silvestri, Parma (IT); Fabrice Euzen, Parma (IT); Guillaume Rolland, Parma (IT)

(73) Assignee: SIDEL S.P.A., 43100 Parma (IT)

(21) Appl. No.: 13/202,542

(22) PCT Filed: Mar. 11, 2009
(86) PCT No.: PCT/IT2009/000093

§ 371 Date: Aug. 19, 2011

(51) Int. Cl. A61L 2/18; A61L 2/16

(52) U.S. Cl. 422/110; 422/292

(57) ABSTRACT

An apparatus for sterilizing objects, comprising: a sterile conduit (2) having an inlet for introducing non sterile objects and an outlet for extracting sterilized objects; spraying means for introducing a sterilization fluid heated and vaporized inside the conduit (2); sucking means (30; 115) for capturing exhaust fluids exiting from the conduit (2). The apparatus is provided with injecting means (10) for injecting a flow of fluid transversely to the conduit (2) to generate a fluid barrier for isolating said sterile conduit from an exterior non sterile area, said fluid barrier being generated at said inlet of the conduit (2). A method for regulating the distribution of a sterilization fluid inside a sterile conduit for sterilizing objects.

### TECHNICAL FIELD AND BACKGROUND ART The present invention relates to an apparatus and a method for sterilizing objects.

As generally known, in most industrial fields and especially in the food industry, namely in the field of aseptic filling of containers with beverages and in the field of aseptic food packaging, there exists a need to sterilize objects, specifically the containers.

Sterilization is generally achieved using chemical agents, e.g. hydrogen peroxide, which can be used on surfaces of every type, such as paper, plastic, metal or organic materials.

With reference to the food industry, in order to ensure that filling takes place in aseptic conditions it is necessary to sterilize not only the containers, both internally and externally, but also their closures, for example caps or stoppers, which serve to seal the containers at the end of the filling process.

In this light, known apparatuses for sterilizing container closures comprise a sterile conduit having a wall defining internally a passage for the closures. Specifically, inside the conduit a volume having a controlled aseptic atmosphere is defined. Said known apparatuses further comprise spraying means for injecting inside the conduit a sterilization fluid (i.e. a chemical agent) heated and vaporized, and drying means for introducing hot air in the conduit. In this view, the apparatus comprises a device for heating and vaporising the sterilization fluid, for instance as described in patent application WO2006128884.

It is noteworthy to observe that the sterilization fluids which are commonly used in these apparatuses are chemically active when their temperature is above a threshold value (activation value), The drying means have the function of activating the sterilization fluid and simultaneously eliminating the sterilization fluid condensed on the closures, before the closures themselves exit the apparatus.

For this reason, the sterilization fluid is also heated before it is injected in vaporized fashion inside the conduit.

The known sterilization apparatuses also comprise a system for the extraction of the exhaust fluids from the conduit, in such a way that they do not flow in the environment.

In the known apparatuses, an exhaust tube is provided, which is connected to a suction pump at one end and directly to the conduit at the opposite end. In this way, a suction force is directly applied to the volume inside the conduit.

More specifically, the exhaust tube is typically connected to the conduit at an opening through which the closures are fed into the conduit for the sterilization process.

The conduit must be kept overpressured with respect to the environment in order to prevent the inflowing of environmental contaminated air inside the aseptic volume.

The known apparatuses have several drawbacks.

First of all, bearing in mind that the conduit must be kept overpressured, a problem arises in relation to the extraction of the exhaust fluids from the conduit. In fact, the suction force applied to the volume inside the conduit establishes a negative pressure gradient next to an end of the conduit wherein the suction pump is connected, typically at an entry of the conduit. Such a negative pressure gradient determines the formation of zones wherein the pressure is less than the environmental pressure outside the conduit, thus inducing the inflowing of contaminated environmental air inside the conduit.

A further problem that arises in relation to the extraction of the exhaust fluids from the conduit is the following. The extraction of exhaust fluids generates undesired flows of the process fluids (i.e. the sterilization fluid and the drying air) inside the conduit, thus altering the distribution of the sterile atmosphere. In other words, the distribution of the process fluids inside the conduit is not optimized because it is affected by the suction force applied at an end of the conduit by the system for extracting the exhaust fluids.

Disadvantageously, the action of the process fluids on the closures is hardly controllable and predictable, due to the fact that the distribution of the process fluids is affected by the extraction of the exhaust fluids.

According to the state of the art, document WO2006/097243 discloses a is device for the sterile filling of liquids in bottles, comprising a steriliser which is used to sterilise the bottles with H₂O₂, a filling element which is used to fill the bottles and a closing element which is used to apply a closing element as a closing lid, means for adjusting the temperature of the bottle such that condensation of the H₂O₂ on the surface of the bottle is prevented, The further prior art document US 3911640 shows a method for packing sterile goods into containers under aseptic conditions wherein a thermoformable web is introduced into one end of a processing tunnel within which sterile conditions are maintained.

According to the prior art, the document DE19945500 discloses a device for filling liquid or paste food in containers, which are previously sterilized by purging with hydrogen peroxide and are then filled within a sterile environment. Sterile air within the machine is directed to create a laminar flow which is employed to seal off the filling chamber (4) with excess air being released to the ambient locality.

The further prior art document US 2007/154343 shows a medical washer for deactivating surfaces of a medical instrument or device having a lumen. The medical washer includes a housing that defines a chamber dimensioned to contain a liquid and to receive a medical instrument having a lumen.

### DISCLOSURE OF THE INVENTION.

An object of the present invention is to eliminate the aforesaid drawbacks and to make available an apparatus for sterilizing objects able to isolate the sterile conduit from the non sterile environment. In particular, an aim of an apparatus according to the invention is to prevent the inflowing of contaminated environmental air inside the sterile volume wherein the objects are sterilized.

A further object of the present invention is to make available an apparatus for sterilizing objects, which provides a capturing of exhaust fluids without affecting the distribution of the process fluids inside the sterile volume wherein the objects are sterilized.

An additional object is to make available an apparatus for sterilizing objects, which is able to optimize the distribution of the process fluids inside the sterile volume wherein the objects are sterilized.

A further object is to propose a method for regulating the distribution of a sterilization fluid inside a sterile volume for sterilizing objects.

Said objects are fully achieved by the apparatus and method for sterilizing objects according to the present invention, which are characterised by the content of the claims set out below.

### BRIEF DESCRIPTION OF THE DRAWINGS

These and other objects shall become more readily apparent from the following description of a preferred embodiment, illustrated purely by way of non limiting example in the accompanying drawing tables, in which:

FIG. 1 shows an apparatus according to the present invention, in a top view;

FIG. 2 shows a schematic lateral view of the apparatus of FIG. 1;

FIG. 3 shows a functional diagram pertaining to the method according to the present invention;

FIG. 4 shows a perspective view of the apparatus of FIG. 1;

FIG. 5 shows a perspective view of injecting means inserted in the apparatus of FIG. 1;

FIG. 6 shows a section view of the injecting means of FIG. 5;

FIG. 7 shows a perspective view of an operative portion of the apparatus of FIG. 1;

FIG. 8 illustrates a perspective view of a delivery pipe arranged inside the operative portion shown in FIG. 7;

FIG. 9 shows a perspective view of sucking means inserted in the apparatus of FIG. 1;

FIG. 10 illustrates an enlarged section view of the sucking means shown in FIG. 9;

FIG. 11 illustrates a further perspective view of the sucking means shown in FIGS. 9 and 10.

With reference to the figures, the numeral 1 globally designates an apparatus for sterilizing objects, in particular container closures such as caps or stoppers, which serve to seal the containers and are applied to the containers at the end of the filling process.

The apparatus 1 comprises a sterile conduit 2 having an inlet 3 for the objects.

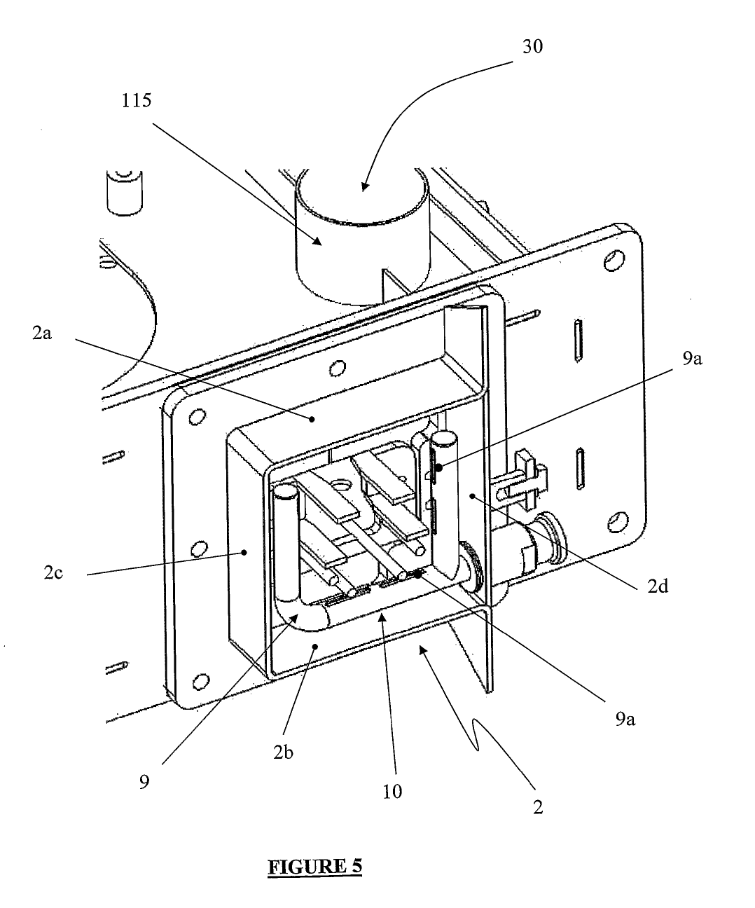

In the preferred embodiment and with specific reference to FIG. 5, the conduit 2 has a rectangular cross-section and it comprises a top wall 2a, a bottom wall 2b and two lateral walls 2c, 2d, each of said walls being substantially flat. In addition, the conduit 2 is preferably arranged along a longitudinal direction.

However, the configuration of the conduit 2 herein described and illustrated is not essential; in fact, the conduit 2 may have a different shape, for example it can be a tube having a circular cross-section and arranged on a curvilinear path.

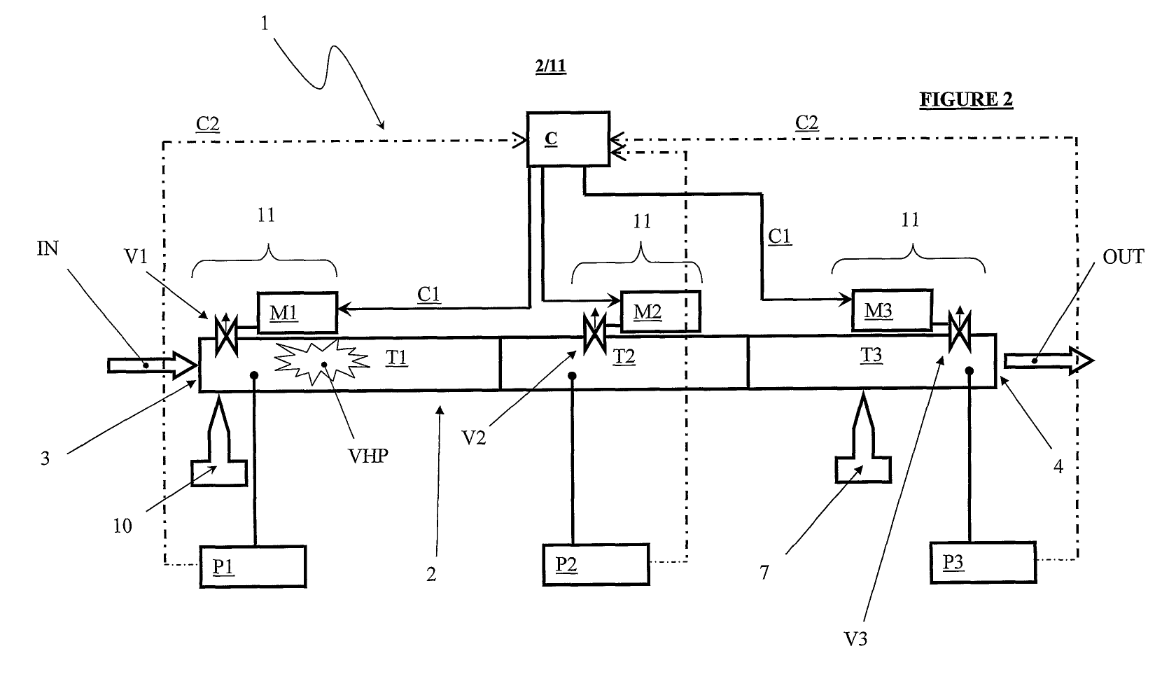

In the preferred embodiment and with particular reference to FIG. 2, a conveyor (not shown in the figures) is arranged inside the conduit 2 to transport the objects from the inlet 3 to an outlet 4 of the conduit 2 itself, said inlet and outlet corresponding to the inlet and outlet of the apparatus 1. The apparatus 1 receives (singularly) the objects to be sterilized at the inlet (see arrows IN) and makes available the sterilized objects at the outlet (see arrows OUT). With particular reference to the container closures, they are preferably arranged inside the conduit 2 with their bottom wall face up and their concave portion face down.

The apparatus comprises a plurality of nozzles coupled to the conduit for injecting a sterilization fluid into the conduit itself. The apparatus is also provided with a system for heating and/or vaporizing the sterilization fluid, in order to feed the nozzles with sterilization fluid which is heated above the activation temperature for that fluid and then vaporized. Such a system, not shown in the figures, is described in the patent application WO2006128884, which is here incorporated by reference.

Therefore, the nozzles, together with said system (i.e. the system for heating and vaporizing the sterilization fluid), define spraying means 5 for introducing a sterilization fluid heated and vaporized inside the conduit 2.

The sterilization fluid is preferably Vapour of Hydrogen Peroxide (VHP).

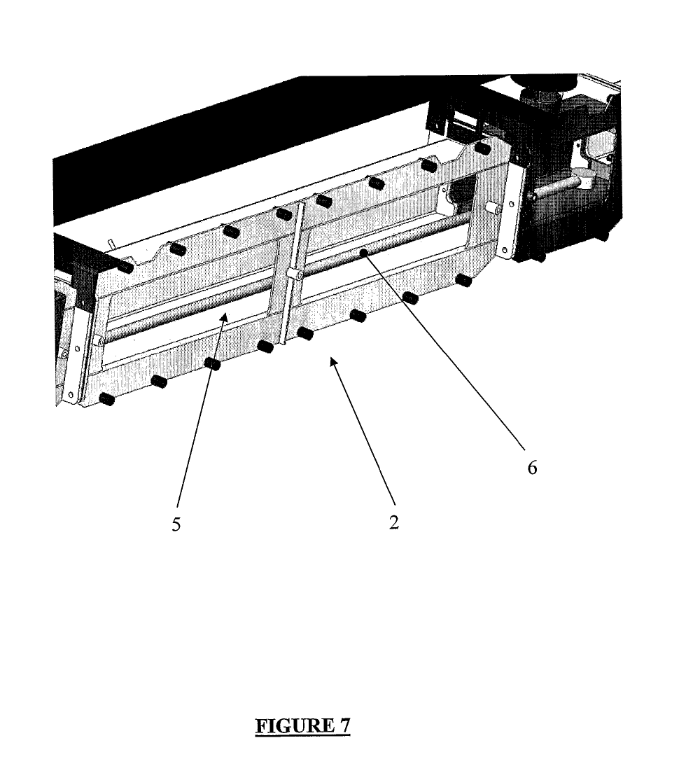

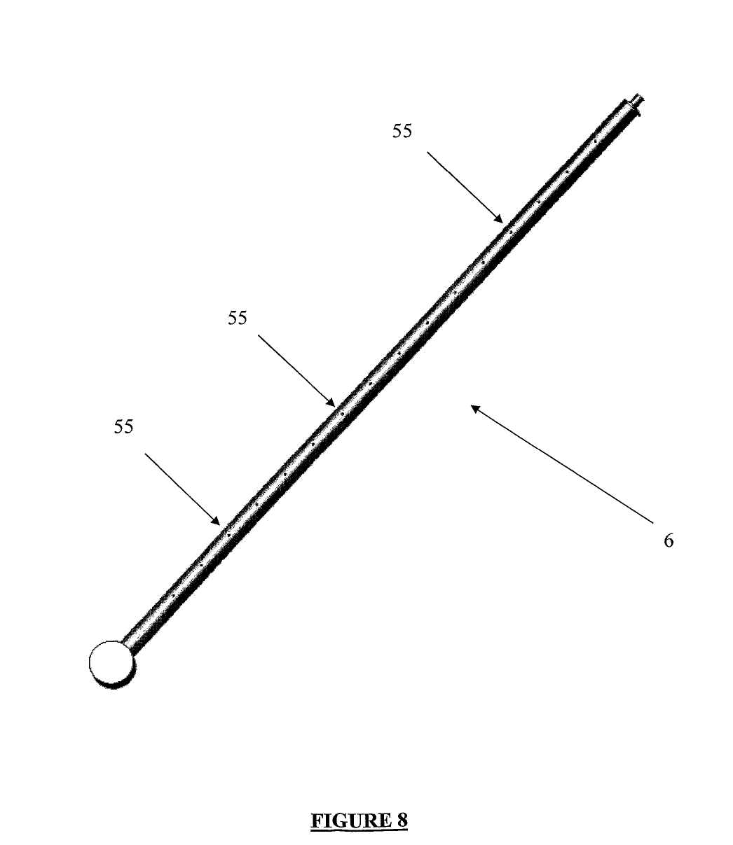

In the preferred embodiment and with specific reference to FIGS. 7 and 8, the spraying means comprise a first delivery pipe 6 arranged inside the conduit 2 and provided with a plurality of holes along its length, said holes defining diffusers 55 which act as nozzles.

With reference to the figures, the apparatus 1 comprises drying means for introducing hot air in the conduit for the purpose of removing the sterilization fluid condensed on the objects.

In the preferred embodiment, said drying means comprise a second delivery pipe, which has a structure identical to that of the first delivery pipe 6 (i.e. is arranged inside the conduit 2), but it is fed with heated air (or any inert gas). As far as the layout of the drying means, being their structure identical to that of said spraying means, reference is made to FIGS. 7 and 8 above mentioned.

The drying means are coupled to the conduit 2 substantially downstream with respect to the spraying means, in the direction of movement of the objects inside the conduit 2, see arrows IN and OUT in FIG. 2.

In this regard, the conduit 2 preferably defines three operative portions T1, T2, T3, which are arranged consecutively along the path followed by the objects in their movement from the inlet 3 to the outlet 4, namely:

a first operative portion T1, wherein the spraying means are active;

a third operative portion T3, wherein the drying means are active;

a second operative portion T2 intermediate between the first and the third operative portions, wherein neither the spraying means nor the drying means are active on the objects.

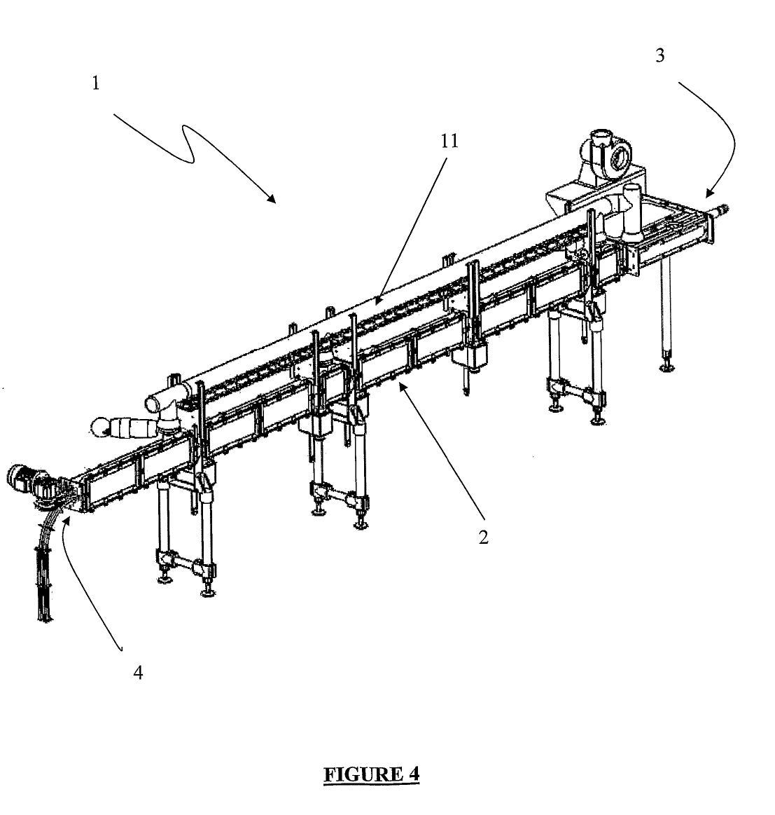

With reference to FIG. 4, the apparatus 1 comprises sucking means 11 for capturing exhaust fluids exiting from the conduit 2.

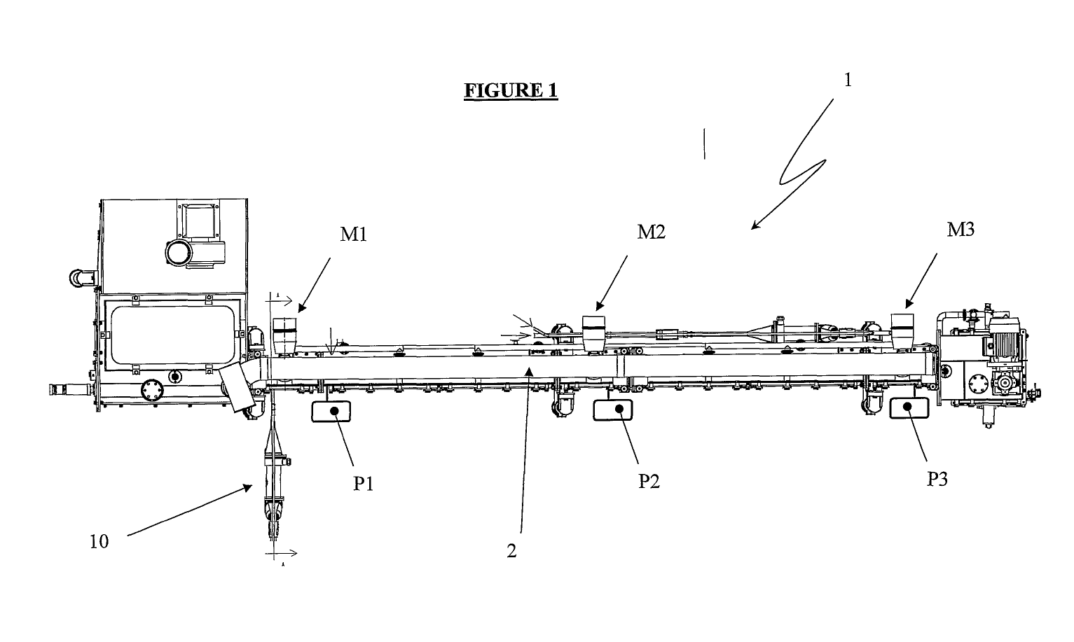

With specific reference to FIGS. 1, 5 and 6, the apparatus comprises injecting means 10 for injecting a flow of fluid transversely to the conduit 2 to generate a fluid barrier for isolating said sterile conduit from an exterior non sterile area, said fluid barrier being generated at said inlet 3 of the sterile conduit is 2. Such a flow of fluid transversely to the conduit generates a strong turbulence in the zone wherein it is injected, thus increasing the pressure in that zone. Due to such an increase of pressure, a sort of plug forms at the inlet of the conduit, thus isolating the sterile conduit from an exterior non sterile area.

In the preferred embodiment, said injecting means 10 are arranged at said inlet of the conduit 2.

In particular, the flow of fluid injected transversely to the conduit 2 generates a blade-shaped fluid barrier. Preferably, such a fluid barrier is made up of hot sterile air.

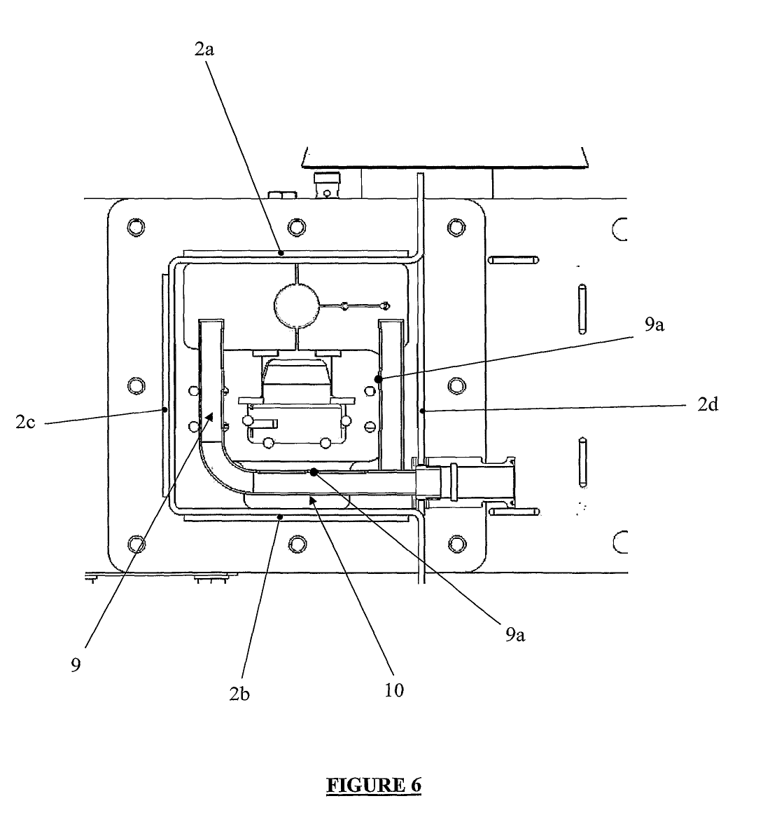

According to the preferred embodiment shown in FIGS. 5 and 6, said injecting means 10 comprise a C-shaped channel 9 arranged on the perimeter of a transversal section of the conduit 2 and having one or more outlets 9a for injecting said flow of fluid generating the fluid barrier. Preferably, the outlets for the injection of the fluid barrier are defined by a plurality of holes or slots distributed along the annular channel.

According to further different embodiments, said injecting means comprise respectively a horizontal channel or an L-shaped channel or an annular channel connected to walls of the conduit, each kind of said channels having one or more outlets for injecting the flow of fluid generating the fluid barrier. Preferably, the outlets for the injection of the fluid barrier are defined by a plurality of holes or slots distributed along the channels.

In the preferred embodiment, the apparatus 1 further comprises:

detecting means for detecting working parameters of the sterile atmosphere;

control means C operatively associated to the sucking means (see lines designated as C1) and to the detecting means (see lines designated as C2), said control means regulating the operation of said sucking means as a function of the detected working parameters.

In the preferred embodiment and with specific reference to FIGS. 1 and 2, the apparatus 1 comprises a plurality of sucking means and a plurality of detecting means all distributed along said conduit at a predetermined distance to each others. Preferably, the detecting means comprise an array of pressure sensors distributed along the conduit 2, typically three pressure sensors P1, P2, P3 one for each of said operative portions T1, T2, T3 of the conduit.

According to a different embodiment, said detecting means comprise, besides the array of pressure sensors, an array of flow sensors distributed along the conduit 2, typically three flow sensors (not illustrated) one for each of said operative portions T1, T2, T3 of the conduit.

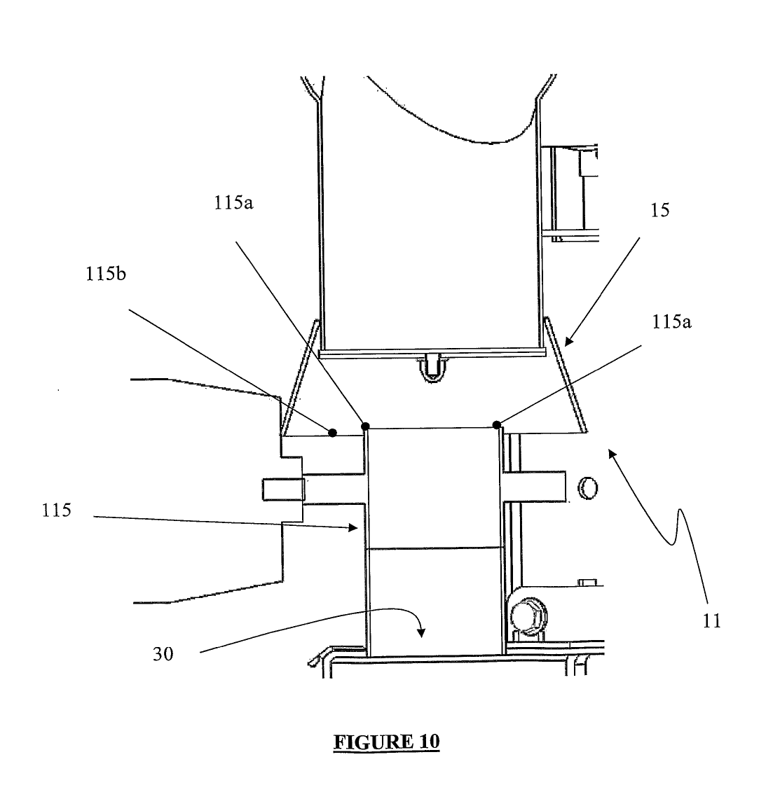

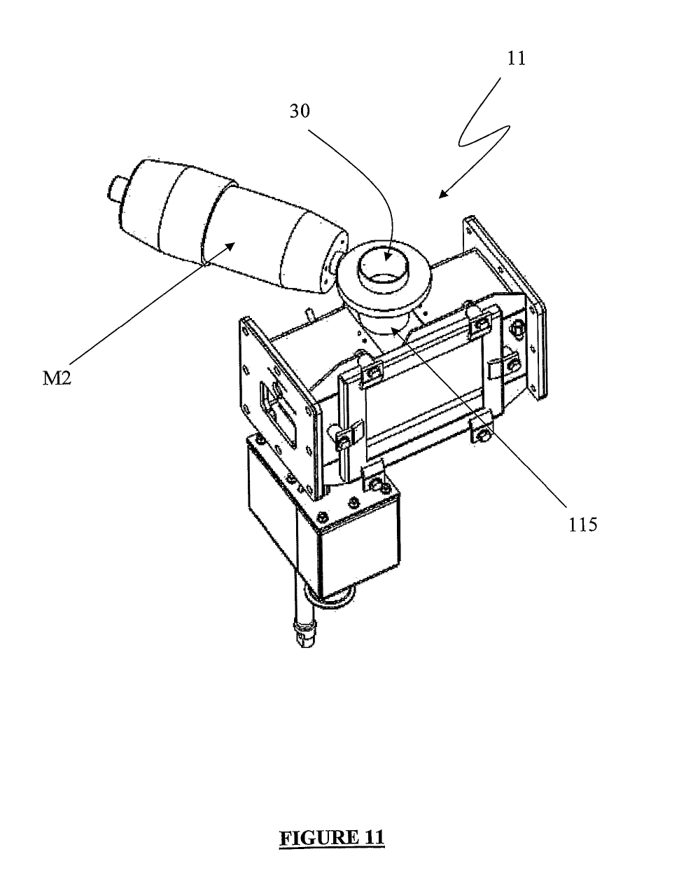

In a further embodiment, the detecting means comprise an array of flow sensors without any pressure sensor. In the preferred embodiment and with particular reference to FIGS. 2 and 9-11, each of said sucking means 11 comprise at least a regulating valve V1, V2, V3 coupled to a corresponding exhaust aperture 30 defined in the conduit 2 and coupled to a respective actuator M1, M2, M3 for activating the operation of said valve between at least a first operative position, wherein the valve closes the exhaust aperture, and a second operative position wherein said valve turns on the exhaust aperture. In addition, each actuator M1, M2, M3 is operatively connected to said control means C and it is triggered as a function of the detected working parameters.

Preferably, each of said sucking means 11 further comprise a suction hood 15 arranged over a corresponding regulating valve V1, V2, V3 coupled to an exhaust aperture 30, said suction hood capturing the exhaust fluids by way of a draught effect.

It is noteworthy to observe that the suction hood 15 capturing the exhaust fluids by way of a draught effect does not affect the distribution of the sterilization fluid inside the sterile conduit, because no suction force is directly applied to the fluid contained inside the conduit 2.

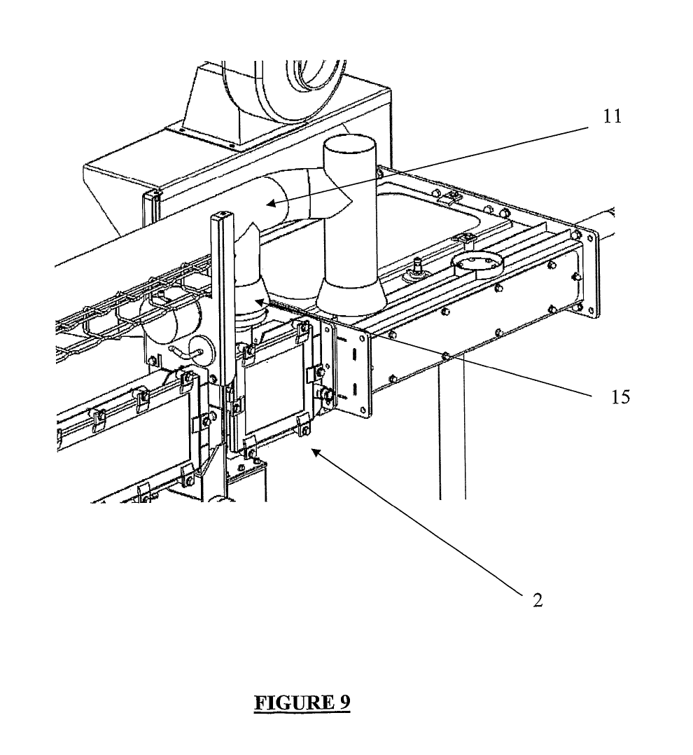

According to the preferred embodiment shown in FIGS. 9-11, said exhaust aperture 30 extends through a hose 115, edges 115a of said hose being prolonged inside a base section 115b of said hood 15, thus preventing outflow of exhaust fluids from the leak between said hood and said hose.

It is further object of the present invention to provide a method for regulating the distribution of a sterilization fluid inside a sterile conduit for sterilizing objects, in particular container closures.

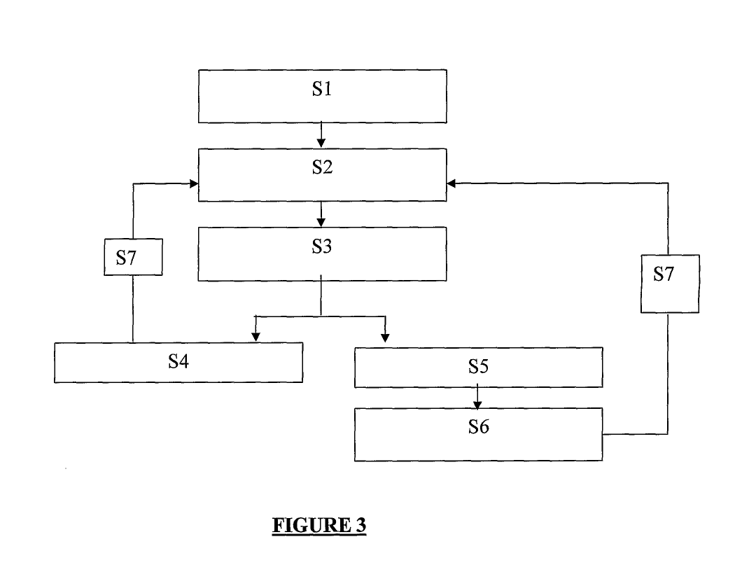

According to the invention, a method for regulating the distribution of a s sterilization fluid inside a sterile conduit for sterilizing objects, as illustrated on FIG. 3, comprises the following steps:

capturing the exhaust fluids through sucking means (box SI);

detecting working parameters of the sterilization fluid inside said sterile conduit (box S2);

comparing the detected working parameters with pre-set values or range of values (box S3);

regulating the operation of said sucking means as a function of the result of said comparing step (boxes S4, S5, S6).

In particular, one of said working parameters is the pressure of the sterile atmosphere and another one is the flow of the sterile atmosphere.

Preferably, the sterilization fluid is Vapour of Hydrogen Peroxide (VHP) heated and vaporized.

The step of regulating the operation of the sucking means as a function of the result of the comparing step is carried out as it follows.

If the detected working parameters coincide with the pre-set values or are within the pre-set range of values, then no regulation of the operation of the sucking means is performed (box S4). On the other hand, if the detected working parameters do not coincide with the pre-set values or are not within the pre-set range of values (box S5), then a regulation of the operation of the sucking means is performed (box S6). In particular, the position of the valve (movable at least between a closing position of the exhaust aperture and a turning on position of said exhaust aperture) is adjusted by the corresponding actuator. The activation of the actuator is triggered by the control means after the step of comparing the detected working parameters with pre-set values or range of values is completed.

The position of the valve determines the amount of exhaust fluid that is captured by the suction hood.

The step of regulating the operation of the sucking means as a function of the result of said comparing step is carried out through a feedback control (see branch designated as S7) based on said working parameters, i.e. the working parameters vary after a first regulation and therefore a potential second regulation is carried out on the basis of a second detection. In this regard, the detecting step is carried out in substantially continuous mode.

The apparatus according to the invention has important advantages.

First of all, an apparatus for sterilizing objects according to the invention is able to isolate the sterile conduit from the non sterile environment in a very simple a reliable manner. In particular, such an apparatus prevents the inflowing of contaminated environmental air inside the sterile conduit wherein the objects are sterilized,

Advantageously, an apparatus according to the invention manages to capture the exhaust fluids without affecting the distribution of the process fluids inside the conduit. Such a result is possible because of the presence of the fluid barrier, the detecting means and the control means.

Advantageously, an apparatus according to the invention manages to optimize the action of the sterilization fluid on the objects. Such a result is possible because of the presence of the detecting means and of the control means.

1-11. (canceled)

12. Apparatus (1) for sterilizing objects, comprising:
a sterile conduit (2) having an inlet (3) for introducing non sterile objects and an outlet (4) for extracting sterilized objects;
spraying means (5) for introducing a sterilization fluid heated and vaporized inside the conduit (2);
sucking means (11) for capturing exhaust fluids exiting from the conduit (2),
characterized in that it comprises injecting means (10) for injecting a flow of fluid transversely to the conduit (2) to generate a blade-shaped fluid barrier for isolating said sterile conduit from an exterior non sterile area, said fluid barrier being generated at said inlet (3) of the conduit (2).

13. Apparatus according to claim 12, wherein said fluid barrier is made up of hot sterile air.

14. Apparatus according to claim 12, wherein said injecting means (10) comprise a horizontal channel connected to a wall of the conduit and having at least an outlet for injecting said flow of fluid generating the fluid barrier.

15. Apparatus according to claim 12, wherein said injecting means (10) comprise a L-shaped channel arranged on walls of the conduit (2) and having at least an outlet for injecting said flow of fluid generating the fluid barrier.

16. Apparatus according to claim 12, wherein said injecting means (10) comprise a C-shaped channel (9) arranged on walls of the conduit (2) and having at least an outlet (9a) for injecting said flow of fluid generating the fluid barrier.

17. Apparatus according to claim 12, wherein said injecting means (10) comprise an annular channel arranged on the perimeter of a transversal section of the conduit (2) and having at least an outlet for injecting said flow of fluid generating the fluid barrier.

18. Apparatus according to claim 12, comprising:
detecting means for detecting working parameters of the sterile atmosphere;
control means (C) operatively associated to the sucking means (11) and to the detecting means, said control means regulating the operation of said sucking means as a function of the detected working parameters.

19. Apparatus according to claim 18, wherein said detecting means comprise at least a pressure sensor (P1, P2, P3).

20. Apparatus according to claim 18, wherein said detecting means comprise at least a flow sensor.

21. Apparatus according to claim 18, wherein said sucking means (11) comprise:
at least one regulating valve (V1, V2, V3) coupled to a corresponding exhaust opening defined in the conduit (2);
said at least one regulating valve (V1, V2, V3) being coupled to a respective actuator (M1, M2, M3) for activating the operation of said valve (V1, V2, V3) between at least a first operative position, wherein the valve closes the exhaust aperture, and a second operative position wherein said valve turns on the exhaust aperture, said actuator being operatively connected to said control means (C) and triggered by the detected working parameters.

22. Apparatus according to claim 21, wherein said sucking means (11) further comprise a suction hood (15) arranged over a corresponding regulating valve coupled to an exhaust aperture (30), said suction hood capturing the exhaust fluids by way of a draught effect.

23. Apparatus according to claim 22, wherein said exhaust aperture (30) extends through a hose (115), edges (115a) of said hose being prolonged inside a base section (115b) of said hood (15).

24. Apparatus according to claim 18, comprising a plurality of sucking means (11) and a plurality of detecting means all distributed along said conduit (2) at a predetermined distance to each others.

25. Apparatus according to claim 12, wherein said injecting means (10) are arranged at said inlet (3) of the conduit (2).
